(19) United States
(12) Patent Application Publication
(10) Pub. No.: US 2020/0276273 A1
(43) Pub. Date: Sep. 3, 2020

(54) METHOD FOR TREATING MYELOID LEUKEMIA

(71) Applicants: UNIVERSITY OF MIAMI, Miami, FL (US); USA, REPRESENTED BY THE DEPT OF VETERANS AFFAIRS, Washington, DC (US)

(72) Inventors: Joaquin J. Jimenez, Miami, FL (US); Andrew Schally, Miami Beach, FL (US); Ren Zhi Cai, Miami, FL (US)

(21) Appl. No.: 16/649,914

(22) PCT Filed: Sep. 20, 2018
(86) PCT No.: PCT/US18/52033

§ 371 Date: Mar. 23, 2020

Related U.S. Application Data
(60) Provisional application No. 62/561,388, filed on Sep. 21, 2017.

(51) Int. Cl. A61K 38/25; A61P 35/02

(52) U.S. Cl. CPC A61K 38/25; A61K 9/0019; A61P 35/02

(57) ABSTRACT

The disclosure provides a method of treating myeloid leukemia, the method comprising administering a GHRH antagonist to mammalian subject in need thereof. The disclosure further provides use of a GHRH antagonist for treating myeloid leukemia or in the preparation of a medicament for treating myeloid leukemia. The disclosure also provides a GHRH antagonist for use in treating myeloid leukemia.

### CROSS REFERENCE TO RELATED APPLICATION

This application claims priority to U.S. Provisional Patent Application No. 62/561,388, filed Sep. 21, 2017, the disclosure of which is hereby incorporated by reference in its entirety.

### GRANT FUNDING DISCLOSURE

This invention was made with government support under grant no. 700203 awarded by the Department of Veteran's Affairs. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 52359_Seqlisting.txt; Size: 11,220 bytes; Created: Sep. 20, 2018.

### FIELD OF DISCLOSURE

The invention relates to materials and methods for treating myeloid leukemia.

### BACKGROUND

Myeloid leukemia (including acute myeloid leukemia and chronic myeloid leukemia) is a hematological malignancy with a poor prognosis. The disease is characterized by the uncontrolled growth of abnormal white blood cells that accumulate in the bone marrow, interfering with the production of normal blood cells. Current therapies consist of chemotherapy, or in severe cases, bone marrow stem cell transplantation. However, traditional approaches are associated with elevated morbidity and mortality rates. In particular, bone marrow transplantation involves risks of Graft-Versus-Host Disease (GVHD) and pulmonary and hepatic complications. Attempts have been made to create targeted therapies for myeloid leukemia, focusing on aberrations in the receptor tyrosine kinase FLT3, but none of these approaches significantly altered the clinical therapy of the disease. Consequently, there remains a need in the art for novel alternative therapeutic approaches for myeloid leukemia.

### SUMMARY

1. The disclosure provides a method of treating myeloid leukemia, the method comprising administering a GHRH antagonist to mammalian subject in need thereof. The disclosure further provides use of a GHRH antagonist for treating myeloid leukemia or in the preparation of a medicament for treating myeloid leukemia. The disclosure also provides a GHRH antagonist for use in treating myeloid leukemia.

2. In various aspects, such as the method or use of paragraph 1, the GHRH antagonist comprises the amino acid sequence (Formula I): R¹-Tyr¹-D-Arg²-Asp³-A⁴-Ile⁵-A⁶-Thr⁷-A⁸-Har⁹A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂ (SEQ ID NO: 2),

wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada(dichloroacetyl-12-aminododecanoyl), Nac (naphthylacetyl), Nac-Ada, Ada-Ada, or CH₃(CH₂)₁₀—CO-Ada;

A⁴ is Ala or Me-Ala;

A⁶ is Cpa (para-chlorophenylalanine) or Phe(F)5;

A⁸ is Ala, Pal (pyridylalanine), Dip ((3,3-diphenyl)alanine), or Me-Ala;

A¹⁰ is FPa5, Tyr(Alk) where Alk is Me or Et;

A¹¹ is His or Arg;

A¹² is Lys, Lys(0-11) (Lys(A0-A1-A2-A3-A4-A5-A6-A7-A8-A9-A10-A11-), Lys(Me)₂, or Orn (ornithine);

A¹⁵ is Abu (alpha-aminobutyric acid) or Orn;

A¹⁷ is Leu or Glu;

A²⁰ is Har (homoarginine) or His;

A²¹ is Lys, Lys(Me)₂ or Orn;

A²⁹ is Har, Arg or Agm (agmatine);

R² is β-Ala, Amc (8-aminocaprylyl), Apa (5-aminopentanoyl), Ada (12-aminododecanoyl), AE2A (8-amino-3,6-dioxaoctanoyl), AE4P (15-amino-4,7,10,13-tetraoxapentadecanoyl), ε-Lys(α-NH2) (a Lys residue, the 8-amino group of which is acylated by the carbonyl group of an N-terminally located amino acid; the α-amino group of the Lys residue is free), Agm (agmatine), or absent; and

R³ is Lys(Oct), Ahx (6-aminohexanoyl), or absent.

3. In various aspects, such as the method or use of paragraph 1, the GHRH antagonist is MIA-602, MIA-604, MIA-606, MIA-610, MIA-640, or MIA-690.

4. In various aspects, such as the method or use of paragraph 1, the GHRH antagonist is MIA-602.

5. In various aspects, such as the method or use of any one of paragraphs 1-4, the GHRH antagonist is administered via intradermal, intramuscular, intraperitoneal, intravenous, intraarterial, subcutaneous, intranasal, epidural, sublingual, intranasal, intracerebral, intraventricular, intrathecal, intravaginal, transdermal, rectally, inhalation, or topical delivery.

6. In various aspects, such as the method or use of paragraph 5, the GHRH antagonist is administered subcutaneously.

7. In various aspects, such as the method or use of any one of paragraphs 1-6, the myeloid leukemia is acute myeloid leukemia.

8. In various aspects, such as the method or use of any one of paragraphs 1-6, the myeloid leukemia is chronic myeloid leukemia.

### BRIEF DESCRIPTION OF THE FIGURES

FIGS. 1A-1C: Decreased proliferation of human AML cell lines expressing GHRH receptors following treatment with GHRH antagonist MIA-602. Expression of GHRH receptors by Western blot (FIG. 1a) in human myelogenous leukemia cell lines KG-1, KG-1a, K-562 and THP-1, and the effect of GHRH antagonist MIA-602 on cell proliferation at (FIG. 1b) 24 hours and (FIG. 1c) 48 hours. The scale bar (b) corresponds to 10 μm. Data points (FIG. 1b-1c) represent average results of four independent experiments per cell line. Cell number count was determined by using MOXI Mini Automated Cell Counter. Results are expressed as means±SEM. ***p<0.001 compared to untreated control cells. GHRH-R: pituitary type GHRH receptor; SV1: GHRH splice variant 1 receptor.

FIGS. 2A-2H: Effect of GHRH antagonist MIA-602 on cell death-related gene expression in human myelogenous leukemia cell lines. Fold-change in expression of cell death-related genes in human myelogenous leukemia cell lines (FIG. 2a) KG-1, (FIG. 2b) THP-1, (FIG. 2c) K-562, and (FIG. 2d) KG-1a following treatment with MIA-602. Cells were incubated in medium containing 50 μM MIA-602 for 24 h, before total RNA was isolated and gene expression assessed by qPCR. Genes with changed expression level over two-fold (p<0.05) are displayed. Effect of GHRH antagonist MIA-602 on protein expression in human myelogenous leukemia cell lines (FIG. 2e-h). Protein levels were analyzed in (FIG. 2e) KG-1, (FIG. 2f) KG-1a, (FIG. 2g) K-562, and (FIG. 2h) THP-1 by Western blot. Cell lysates were prepared after incubation with 5 μM MIA-602 for the indicated time points. Beta-actin is shown as the loading control.

FIG. 3A-3F: Xenografted tumors of human AML cell lines KG1a, K562, and THP1 express GHRH receptors and respond to treatment with GHRH antagonist MIA-602. The expression of GHRH ligand and receptors by Western blot (FIG. 3a). Tumors were harvested from untreated animals 4 weeks after injection of leukemia cells were subjected to western blot and immunohistochemical analysis. Scale bar (a) corresponds to 100 μm. The effect of treatment with antagonist MIA-602 on the growth of (FIG. 3b) KG-1, (FIG. 3c) KG-1A, (FIG. 3d) K-562, and (FIG. 3e) THP-1 tumors. Mice were treated with MIA-602 (10 μg, b.i.d.) (open symbol) and compared to non-treated, control animals (filled symbol). Results are expressed as means±SEM, n=15 mice per group, with the exact tumor volumes shown in (FIG. 3f).

FIGS. 4A & 4B: GHRH receptors expressed in mononuclear cells derived from bone marrow aspirates of patients with AML. Expression of GHRH receptors by Western blot (FIG. 4a) in primary leukemia samples (AML1-9). Expression was also determined by immunohistochemistry. Demographics data for these patients is indicated in (FIG. 4b). Patients' leukemia was classified according the updated WHO classification of hematological malignancies (Arber, Blood 127, 2391-405 (2016)): AML1 AML with t(9;11)(p22;q23) MLLT3-KMT2A and t(4;15)(p31;q22) TMEM154-RASGRF1; AML2-AML with IDH1 (132R/C) and DNMT3A mutations; AML3-AML minimally differentiated with trisomy 13; AML4-AML with NRAS and TET2 mutations, AML5-AML with NPM1 mutated; AML6-AML with FLT3-ITD duplication; AML7-AML with NRAS mutation; AML8-AML with IDH2 (140R/Q) mutation; AML9-AML with CSF3R & TET2 mutations.

### DETAILED DESCRIPTION

The disclosure provides a method of treating myeloid leukemia (e.g., acute myeloid leukemia or chronic myeloid leukemia). The method comprises administering a GHRH antagonist to mammalian subject in need thereof. The data set forth herein reveals the presence of GHRH-R on three human AML cell lines, K-562, THP-1, and KG-1a, and demonstrates that GHRH antagonists (e.g., MIA-602) significantly inhibit proliferation of these cells. Treatment with a GHRH antagonist (MIA-602) drastically reduced growth of these human AML cell lines xenografted into nude mice. Furthermore, the expression of GHRH-R was confirmed in nine of nine samples from patients with AML. Targeting GHRH receptors is a new therapeutic approach to myeloid leukemia.

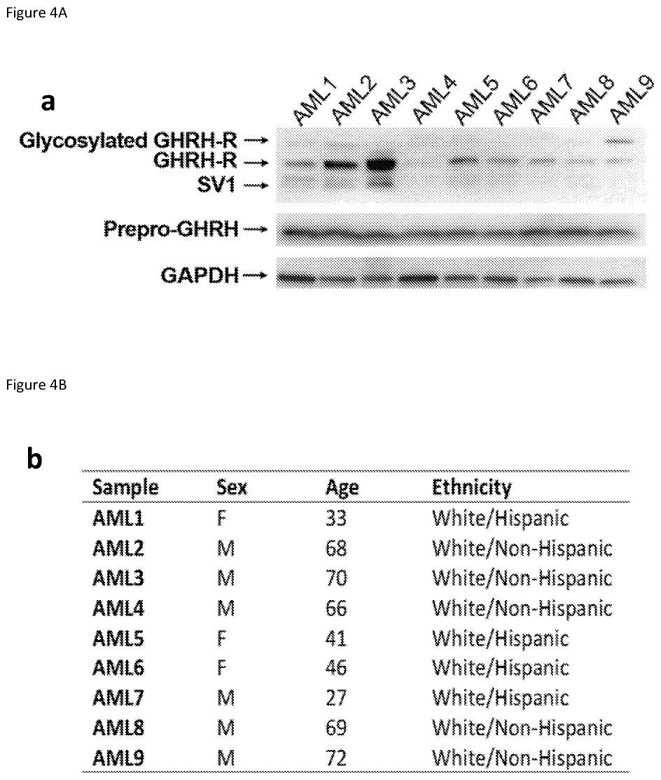

The term “subject” includes, but is not limited to, human and non-human mammals such as wild, domestic and farm animals. Preferably, the subject is a human. The subject may be suffering from any form of myeloid leukemia (acute myeloid leukemia or chronic myeloid leukemia or related neoplasms) as referenced in the World Health Organization (WHO) classification of myeloid neoplasms and acute leukemia. The relapsed or refractory state of the disease is contemplated. The forms of AML are described above in reference to FIG. 4.

Growth hormone-releasing hormone (GHRH) is secreted by the hypothalamus and acts on the pituitary gland to stimulate the release of growth hormone (GH). Nearly 2000 synthetic antagonistic analogs of GHRH have been produced by amino acid substitutions in the biologically active N-terminal of GHRH (1-29). Schally et al., Nat. Clin. Pract. Endocrinol. Metab. 4 (1), 33-43 (2008); Zarandi et al., PNAS 91 (25), 12298-302 (1994); Zarandi et al., Peptides 89, 60-70 (2017). The pituitary GHRH receptor (pGHRH-R) is a seven-transmembrane-domain receptor coupled to G-protein. Rekasi et al., PNAS 97 (19), 10561-6 (2000); Havt et al., PNAS 102 (48), 17424-9 (2005). The pGHRH-R, as well as its truncated splice variants (SV) is expressed in various human tissues. SV1 differs from pGHRH-R in the amino-terminal extracellular domain. Rekasi, supra.

In various aspects, the GHRH antagonist is a peptide. Various modifications of GHRH peptides confer antagonistic properties. The GHRH fragment comprising residues 1 to 29, or GHRH(1-29), is the minimum sequence necessary for biological activity on the pituitary. This fragment retains 50% or more of the potency of native GHRH. Many synthetic analogs of GHRH, based on the structure of hGH-RH(1-29)NH₂ peptide have been prepared are contemplated herein for use in the context of the method. hGHRH(1-29)NH₂ has the following amino acid sequence: Tyr-Ala-Asp-Ala-Ile⁵-Phe-Thr-Asn-Ser-Tyr¹⁰-Arg-Lys-Val-Leu-Gly¹⁵-Gln-Leu-Ser-Ala-Arg²⁰-Lys-Leu-Leu-Gln-Asp²⁵-Ile-Met-Ser-Arg²⁹-NH₂ (SEQ ID NO: 1). The GHRH antagonist may comprise a GHRH peptide sequence to which amino acid deletions, insertions, and/or substitutions have been made. The GHRH antagonist may also be a fragment or modified fragment of GHRH having the capability to bind to the GHRH receptor and inhibiting the release of growth hormone. These antagonistic properties are believed to result from replacement of various amino acids and acylation with aromatic or nonpolar acids at the N-terminus of GHRH(1-29)NH₂.

Optionally, the GHRH antagonist is an antagonist described in U.S. Patent Publication No. 20150166617 (incorporated by reference herein in its entirety and particularly with respect to description of GHRH antagonists). For example, in various embodiments, the GHRH antagonist comprises the amino acid sequence (Formula I/SEQ ID NO: 2): R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂, wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada(dichloroacetyl-12-aminododecanoyl), Nac (naphthylacetyl), Nac-Ada, Ada-Ada, or CH₃(CH₂)₁₀—CO-Ada; A⁴ is Ala or Me-Ala; A⁶ is Cpa (para-chlorophenylalanine) or Phe(F)₅; A⁸ is Ala, Pal (pyridylalanine), Dip ((3,3-diphenyl)alanine), or Me-Ala; A¹⁰ is FPa5, Tyr(Alk) where Alk is Me or Et; A¹¹ is His or Arg; A¹² is Lys, Lys(0-11) (Lys(A0-A1-A2-A3-A4-A5-A6-A7-A8-A9-A10-A11-), Lys(Me)₂, or Orn (ornithine); A¹⁵ is Abu (alpha-aminobutyric acid) or Orn; A¹⁷ is Leu or Glu; A²⁰ is Har (homoarginine) or His; A²¹ is Lys, Lys(Me)₂ or Orn; A²⁹ is Har, Arg or Agm (agmatine); R₂ is β-Ala, Amc (8-aminocaprylyl), Apa (5-aminopentanoyl), Ada (12-aminododecanoyl), AE₂A (8-amino-3,6-dioxaoctanoyl), AE₄P (15-amino-4,7,10,13-tetraoxapentadecanoyl), ε-Lys(α-NH₂) (a Lys residue, the 8-amino group of which is acylated by the carbonyl group of an N-terminally located amino acid; the α-amino group of the Lys residue is free), Agm (agmatine), or absent; and R³ is Lys(Oct), Ahx (6-aminohexanoyl), or absent.

Optionally, the GHRH antagonist is MIA-602: [PhAc-Ada⁰-Tyr¹, D-Arg², Fpa5⁶, Ala⁸, Har⁹, Tyr(Me)¹⁰, His¹¹, Orn¹², Abu¹⁵, His²⁰, Orn²¹, Nle²⁷, D-Arg²⁸, Har²⁹]hGH-RH(1-29)NH₂, further described in U.S. Patent Publication No. 20150166617 (incorporated herein by reference with respect to the discussion of the structure, activity, and methods of making MIA-602, MIA-604, MIA-606, MIA-610, MIA-640, and MIA-690). Alternative GHRH antagonists include, but are not limited to, Phac-Ada-Tyr¹-D-Arg²-Asp³-Ala⁴-Ile⁵-Phe(F)₅-⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala-¹⁹-His²⁰-Orn²¹-Leu²²-Leu²³-Gln²⁴-Asp²⁵Ile²⁶-Nle²⁷-D-Arg²⁸-Har²⁹-Agm-NH₂ (MIA-604/SEQ ID NO: 3); Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606/SEQ ID NO: 4); Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-610/SEQ ID NO: 5); Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Glu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-640/SEQ ID NO: 6); Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690/SEQ ID NO: 7). The amino acid sequences of the peptides described above are numbered in correspondence with the amino acid residues in hGHRH(1-29) (SEQ ID NO: 1).

The disclosure provides a method of treating myeloid leukemia in a subject in need thereof. “Treating” myeloid leukemia does not require a 100% remission. Any decrease in leukemia cells or symptoms of myeloid leukemia constitutes a beneficial biological effect in a subject. The progress of the method in treating myeloid leukemia can be ascertained using any suitable method, such as complete blood counts and/or bone marrow analysis. In certain aspects, the method provides a reduction in the amount of myeloid leukemia cells by at least 50%, at least 65%, at least 75%, at least 80%, at least 85%, at least 90%, at least 95% or by at least 99% as compared to a reference sample, i.e., a sample taken from the subject prior to treatment or a sample taken from a leukemia patient with similar burden but not exposed to the antagonist. In various aspects, “treatment” also includes stabilization of the disease, i.e., controlled or no further progression of the disease (e.g., amount of leukemia cells does not increase, or increases by less than 10%, preferably less than 5%, within a given timeframe).

A particular administration regimen for a particular subject will depend, in part, upon the amount of antagonist administered, the route of administration, and the cause and extent of any side effects. The amount administered to the subject (e.g., human) in accordance with the disclosure should be sufficient to affect the desired response (i.e., ameliorate, prevent or improve an unwanted condition, disease or symptom of a patient) over a reasonable time frame. A therapeutically effective amount of the GHRH antagonist is typically an amount such that when it is administered in a physiologically tolerable excipient composition, it is sufficient to achieve an effective systemic concentration or local concentration in target tissue.

The dose of GHRH antagonist is optionally about 0.005 mg/kg to about 100 mg/kg. In various aspects, the GHRH antagonist is administered in a dose of about 0.05 mg/kg to about 20 mg/kg. In some embodiments, the GHRH antagonist is administered at a dose of about 0.01 mg/kg/dose to about 50 mg/kg/dose, about 0.01 mg/kg/dose to about 25 mg/kg/dose, about 0.1 mg/kg to about 15 mg/kg, or about 1 mg/kg to about 10 mg/kg. Optionally, doses are given once a day or divided into 2-4 administrations/day. When the GHRH antagonist is administered intravenously to human patients, doses are optionally divided into 1-4 bolus injections/day or given as a continuous infusion.

The GHRH antagonist may be administered daily, at least once a week, at least twice a week, at least three times a week, at least four times a week, at least five times a week, six times a week, every two weeks, every three weeks, every four weeks, every five weeks or every six weeks. The treatment period (entailing multiple administrations of the antagonist) will depend on the nature and severity of the disease, as well as the existence of any side effects. Examples of treatment periods include, but are not limited to, 2 weeks, 3 weeks, 1 month, 2 months, 3 months, 4 months, 5 months, 6 months, 9 months, and 12 months.

Methods of administration may include, but are not limited to, oral administration and parenteral administration, including but not limited to, intradermal, intramuscular, intraperitoneal, intravenous, intraarterial, subcutaneous, intranasal, epidural, sublingual, intranasal, intracerebral, intraventricular, intrathecal, intravaginal, transdermal, rectally, by inhalation, or topically (e.g., to the ears, nose, eyes, or skin). The antagonist is preferably administered subcutaneously.

Optionally, the GHRH antagonist is administered either alone or in combination (concurrently or serially) with other pharmaceuticals. In some aspects, the method comprises administering multiple GHRH antagonists. Alternatively or in addition, the GHRH antagonist is optionally administered in combination with other anti-cancer or anti-neoplastic agents. In this regard, in various aspects, the method comprises further administering to the subject one or more chemotherapeutic agents, such as alkylating agents (e.g., cyclophosphamide and ifosfamide), antimetabolites (e.g., cytarabine, 5-fluorouracil, and methotrexate), antitumor antibiotics (e.g., adriamycin and mitomycin), plant-derived anticancer agents (e.g., vinblastine, vincristine, vindesine, and Taxol), cisplatin, carboplatin, and etoposide. Alternatively or in addition, the GHRH antagonist is part of a therapeutic regimen involving cancer therapies other than chemotherapy, such as, for example, surgery or radiotherapy. In some embodiments, the GHRH antagonist is administered in combination with antibiotics, optionally as a single, combined formulation or as separate compositions.

The GHRH antagonist may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred antagonists are salts of low solubility, e.g., pamoate salts and the like.

Formulations containing the GHRH antagonist and a suitable carrier can be solid dosage forms which include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and powder. In some embodiments, a single dose may comprise one or more administrations (i.e., multiple injections or multiple pills to arrive at a single dose/amount of antagonist).

The GHRH antagonist may be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted. Pharmaceutical compositions can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The invention, thus generally described, will be understood more readily by reference to the following example, which is provided by way of illustration and is not intended to limit the invention.

### Examples

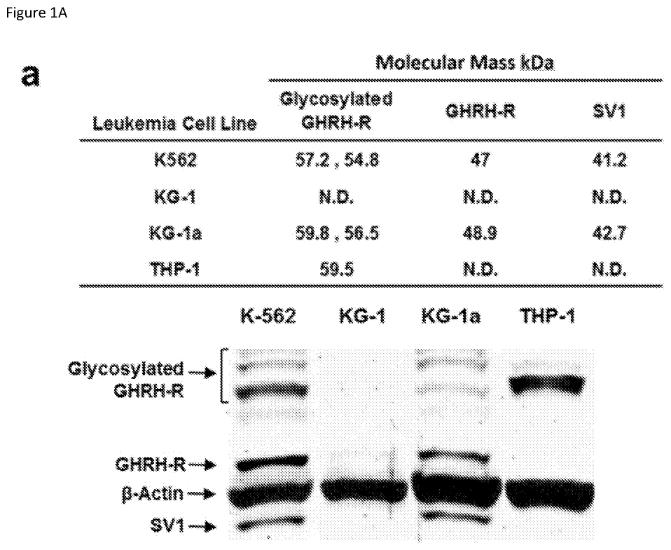

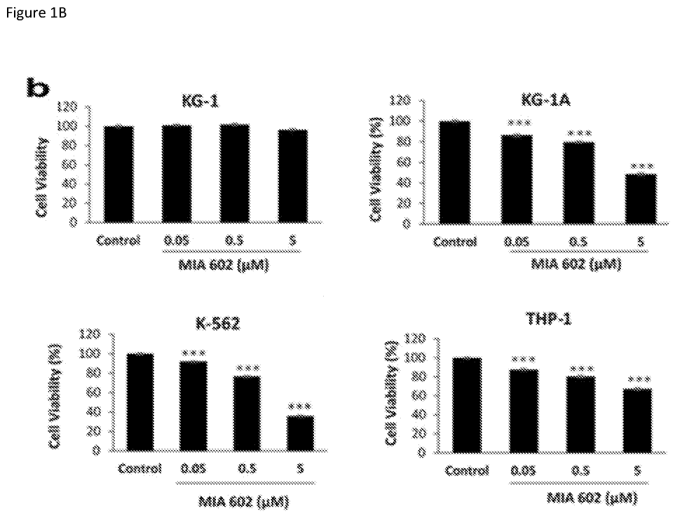

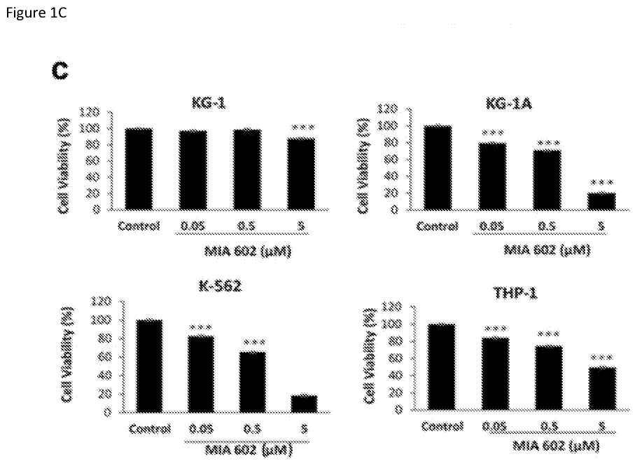

The effects of GHRH antagonist MIA-602 was examined in four human acute myeloid leukemia lines both in vitro and in vivo. Expression of pGHRH-R and its SV1 variant was observed in human leukemic cell lines. K562, THP-1, KG-1, and its subclone KG-1a are established models of acute myelogenous leukemia. Andersson et al., Int. J. Cancer 23 (2), 143-7 (1979); Koeffler et al., Blood 56 (3), 344-50 (1980); Collins et al., Nature 270 (5635), 347-9 (1977); Tsuchiya et al., Int. J. Cancer 26 (2), 171-6 (1980). K-562 expressed both the pituitary type and SV1 receptors, THP-1 cells only pGHRH-R, the subclone KG-1a both SV1 and low levels of pGHRH-R, and the parental KG-1 cell line neither type [FIG. 1a]. This expression pattern was confirmed by immunocytochemistry.

KG-1, KG-1a, K-562, and THP-1 cells were cultured in the presence of 0.05 μM to 5 μM MIA-602 for 24 and 48 hours. Treatment with MIA-602 induced a significant dose- and time-dependent reduction in cell proliferation in the three GHRH-R-positive cell lines, but not in KG-1. Thus, MIA-602 induced a robust decrease in cell proliferation at 5 μM concentration, which was over 50% at 24 hours, and approximately 80% at 48 hours in K-562 and KG-1a cells [FIG. 1b, 1c]. The reduction in cell proliferation was smaller in THP-1 cells which express only the pituitary-type receptor, amounting to 32.7% at 24 hours and 50.4% at 48 hours. Cell growth in the GHRH-R-negative KG-1 line was not changed by MIA-602 at 24 hours and only slightly decreased after 48 hours [FIG. 1c]. Notably, the relative reduction in the proliferation rate in KG-1a suggested that blocking SV1 with the antagonist MIA-602 produces a superior anti-proliferative effect than antagonizing the pGHRH-R alone.

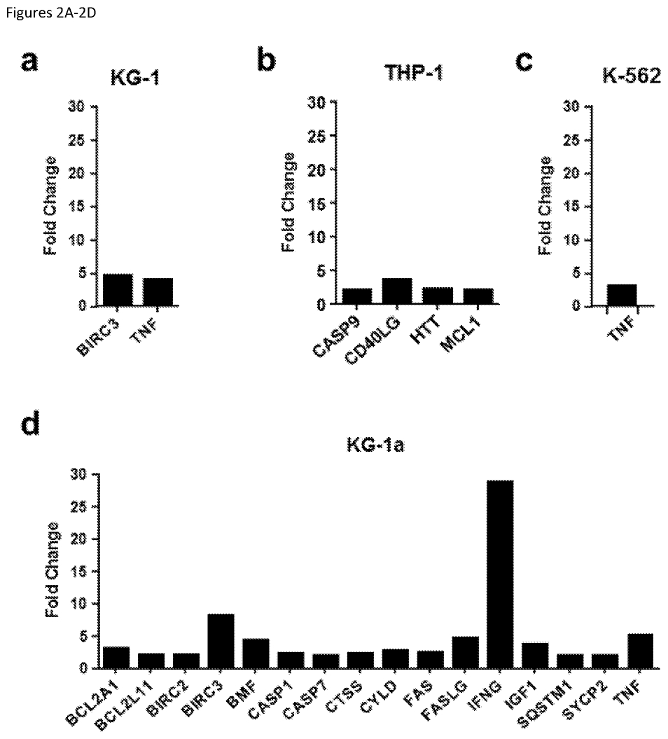

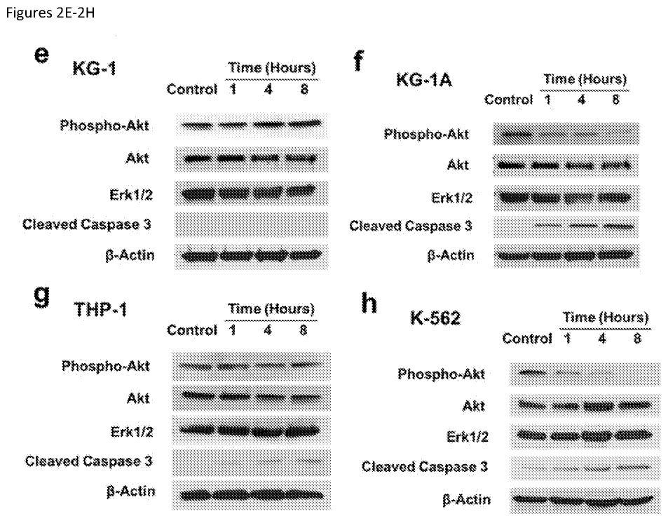

Using a panel of 84 death-related genes, it was observed that the GHRH antagonist MIA-602 functions in these cell lines principally by activating pro-apoptotic pathways. In KG-1a cells, MIA-602 induced significant changes (≥2 fold; p<0.05) in the expression level of 16 genes death-related genes; in particular, pro-apoptotic gene IFNG was upregulated 28.87-fold (p<0.05) [FIG. 2d]. MIA-602 also significantly increased expression of CASP9 (2.08-fold; p<0.05), CD40LG (3.72-fold; p<0.05), HTT (2.25-fold; p<0.05), and MCL1 (2.15-fold; p<0.01) [FIG. 2b] in THP-1 cells. Although the first three genes (CASP9, CD40LG, and HTT) are more pro-apoptotic in nature, only select isoforms of the MCL1 protein encourage apoptosis. In K-562 cells, the GHRH antagonist induced the upregulation of the pro-apoptotic gene TNF (2.16-fold; p<0.01) [FIG. 2c]. Collectively, these findings suggest that MIA-602 reduces the proliferation of human myelogenous leukemic cell lines by upregulating pro-apoptotic genes, although autophagy may be also activated.

MIA-602 also induced changes in gene expression in the KG-1 cell line. After 24-hour of treatment with MIA-602, there was a significant increase in the expression of both BIRC3 (4.64-fold; p<0.01) and TNF (4.11-fold; p<0.01) [FIG. 2a]. Probably this result is due to the heterogeneity of cancer cell populations, since at least one KG-1 subclone (i.e., KG-1a) responds to GHRH-R blockade. Therefore, some response to MIA-602 could result from subclones within the KG-1 population or other mechanisms.

Protein expression analyses in the GHRH-R positive K562, THP-1, and KG-1a lines revealed that phosphorylation of the serine/threonine protein kinase Akt after 8 h incubation with MIA-602 is decreased by 100% in K-562, 85% in KG-1a, and 16% in THP-1 [FIG. 2e-h]. A concomitant increase in cleaved caspase 3 was also observed, indicating activation of the apoptotic pathway. Akt regulates cellular apoptosis through the phosphorylation and inhibition of pro-apoptotic members of the Bcl-2 protein family. None of these changes were found in the KG-1 cells. Moreover, cellular uptake of fluorescently labeled MIA-602 was not observed in KG-1 cells. While not wishing to be bound to any particular theory, the anti-proliferative effect of GHRH antagonist MIA-602 could be mediated by inactivation of Akt, which in turn promotes cell apoptosis.

The percentage of apoptotic cells after 48-h treatment with 5 μmol/l MIA-602 was also measured by TUNEL. For THP-1 cells, untreated cells showed 2% apoptosis (SEM=1%), whereas MIA-602-treated showed 51% (SEM=2 5%). For both KG1a cells and K562 cells, apoptosis in the control condition was 1% (SEM=0 5%) and between 76 and 77% (SEM=3% for KG1a and 3-5% for K562) in MIA-602-treated cells. For the KG1 cells, the control condition showed 1% apoptosis (SEM=0-3%)—similar to the other cell lines when untreated—but the MIA-602 treatment control showed only 12% (SEM=2%).

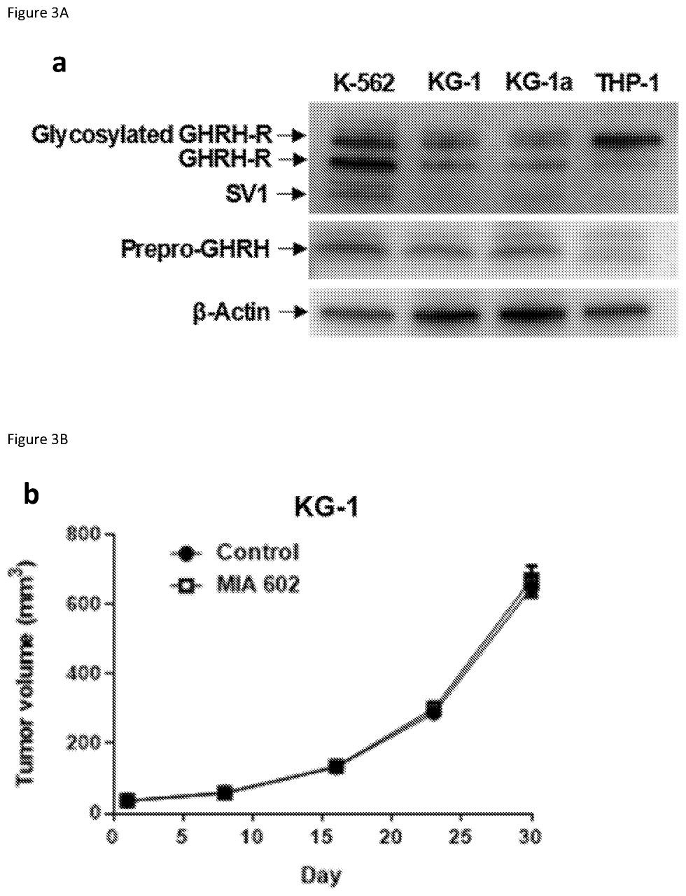

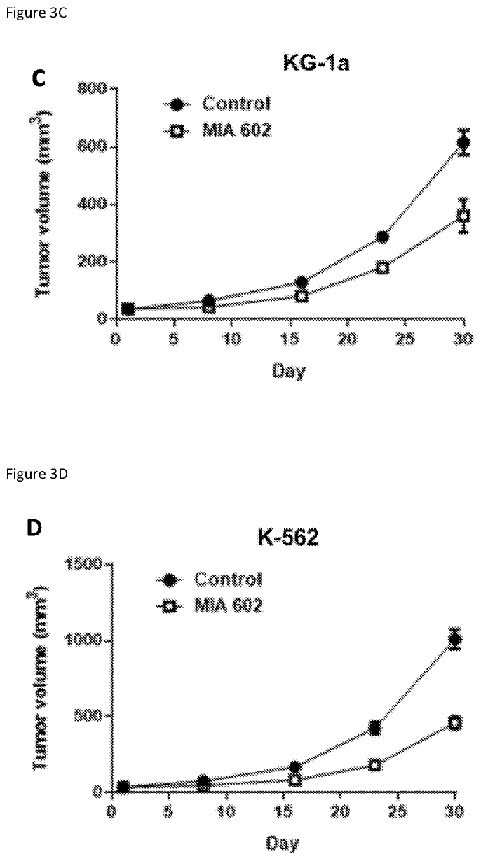

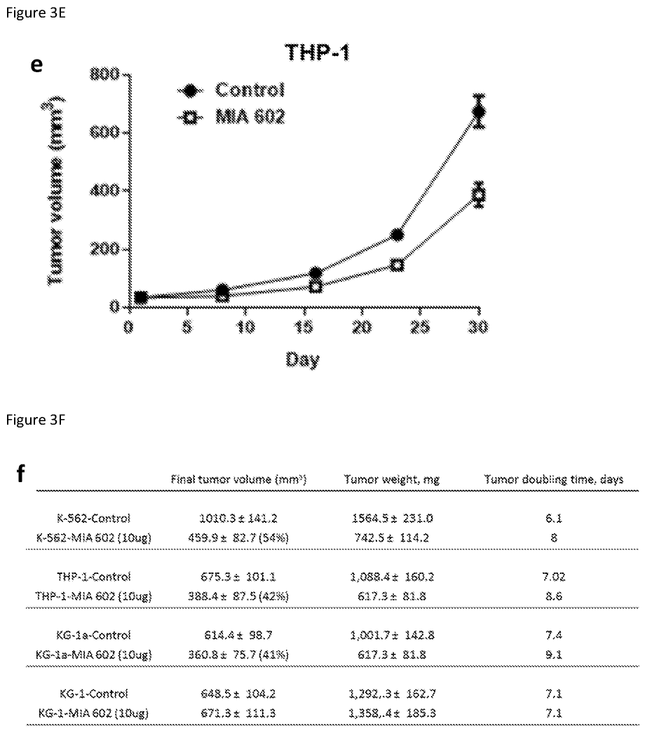

To confirm the efficacy of treatment with GHRH antagonist, in vivo the cell lines were xenografted by subcutaneous injection into athymic nude mice. Western blot analysis and immunohistochemistry of tumors from untreated animals showed that, GHRH ligand was expressed in all tumors, with the highest levels in K-562 and KG-1a tumors, while in THP-1 and KG-1 tumors SV1 receptor was absent. p-GHRH-R was detected in KG-1 cells by Western blot but not by immunohistochemical analysis. MIA-602 was administered for 30 days at a dose of 10 μg B.i.d. Therapy with MIA-602 significantly (P<0.05) decreased the final volume of K-562, THP-1, and KG-1a tumors by 54%, 42%, and 41%, respectively, and prolonged the tumor doubling time [FIG. 3A-F]; but, no changes occurred in the negative control (KG-1) tumor volume. No significant differences in body weights or weights of various organs (liver, spleen, and kidneys) were observed between treated animals and those bearing no tumors. Macroscopic observation of organs revealed no differences after treatment with MIA-602.

Finally, to assess the translational applicability of these results, primary cell samples from AML patients were analyzed for the expression of GHRH receptors. Immunofluorescent staining on the mononuclear cells indicated positive expression of the GHRH receptor in all samples. Western blotting similarly showed positive expression of the pituitary type of GHRH receptor on 9/9 samples from AML patients, as well as expression of the SV1 receptor variant in 8/9 samples [FIG. 4a]. These results provide strong support for the approach to AML based on targeting of GHRH-R.

While not wishing to be bound by any particular theory, the repressive effects of GHRH antagonists on tumors appear to be due in part to the inhibition of the pituitary GH/hepatic IGF-I axis, suppression of autocrine production of IGF-I in tumors, and the blocking of the action of tumoral GHRH1; but, the inhibition of binding and hence of the action of autocrine GHRH produced in tumors and the downregulation of GHRH receptors are most likely to constitute the main effects of GHRH antagonist. MIA-602 has been also shown to decrease Pak1-mediated expression of both pSTAT3 and p65 in gastric cancer cell lines and xenografted tumors. Gan et al., PNAS 113 (51), 14745-14750 (2016). This effect on Pak1-Stat3/NF-kB signaling may underlie the pro-apoptotic effects on AML cell lines. GHRH antagonists have important advantages over cytotoxic compounds, by being devoid of toxic side effects and by the capability to be used for targeted therapy to GHRH receptors on tumors. GHRH antagonists could be also used in combination with chemotherapeutic agents to augment their antitumor effects.

This study reports a novel approach to the treatment of AML based on antagonists of GHRH. The targeted nature of this approach has the advantage of involving minimal toxicity in contrast to the risks associated with chemotherapy and transplantation. This novel strategy based on targeted antagonists of GHRH could provide a useful approach to AML, alone or in combination with traditional intervention.

The results described herein were generated using the following methods.

Peptides and Reagents:

GHRH antagonist MIA-602 was synthetized by solid phase method and purified by reversed-phase high performance liquid chromatography (HPLC) as described previously. Zarandi et al., PNAS 91 (25), 12298-302 (1994); Zarandi et al., Peptides 89, 60-70 (2017). The chemical structure of MIA-602 is [PhAc-Ada)⁰-Tyr¹, D-Arg², Fpa5⁶, Ala⁸, Har⁹, Tyr(Me)¹⁰, His¹¹, Orn¹², Abu¹⁵, His²⁰, Orn²¹, Nle²⁷, D-Arg²⁸, Har²⁹]hGH-RH(1-29)NH₂. Non-coded amino acids and acyl groups are abbreviated as follows: Abu, alpha-aminobutyric acid; Ada, 12-aminododecanoyl; Fpa5, pentafluoro-phenylalanine; Har, homoarginine; Nle, norleucine; Orn, ornithine; PhAc, phenylacetyl; Tyr(Me), O-methyl-tyrosine. The peptide was purified by HPLC before use. For in vitro studies, the peptide was dissolved in dimethyl sulfoxide (DMSO) and diluted with incubation media. Final concentration of DMSO did not exceed 0.1%. For in vivo studies, MIA-602 was dissolved in DMSO and diluted with sterile aqueous 10% phosphate-buffered saline (PBS) solution used also as vehicle control.

Cell Culture:

Human myeloid leukemic cell lines KG-1, KG1a, K-562, and THP-1 were obtained from American Type Culture Collection (ATCC) and cultured in IMDM and RPMI 1640 medium, respectively. KG-1 cell line is a myeloblast derived from human bone marrow macrophage. This cell line is EBNA negative and express HLA A30, A31, B35, and CW4 antigens. KG-1a is an EBNA negative sub-clone of KG-1 cell line after 35 passages. The variant KG-1a is composed of undifferentiated promyeloblasts. The K-562 is an EBNA negative cell line derived from CML patient in terminal blast crises. K-562 is a human erythroleukemia line. THP-1 is a cell line derived from human peripheral blood monocyte of a 1-year old infant with acute monocytic leukemia. The THP-1 cell line express HLA A2, A9, B5, DRw1 and DRw2 antigens. Kg-1, KG-1a and K-562 were maintained in IMDM medium, while THP-1 cells were cultured in RPMI-1640 medium. Both media were supplemented with 2 mM L-Glutamine, 25 mM HEPES, 10% FBS, and 50 μg/ml Gentamicin. Cells were cultured in a 5% CO₂ incubator with 98% humidity at 37° C.

Cell Proliferation Study:

Human myelogenous leukemia cell lines KG-1, KG-1a, K-562 and THP-1 were seeded onto 12-well microplates (Falcon) at a density of 2.5×10⁵ cells/mL in their corresponding media supplemented with 2 mM L-Glutamine, 25 mM HEPES, 1% FBS, and 50 μg/ml Gentamicin. Cells were treated with MIA-602 at concentrations of 0.05, 0.5, and 5 μM for 24 and 48 hours. Each treatment was performed in quadruplicate. Cell proliferation was evaluated by using the MOXI Mini Automated Cell Counter.

Total RNA Isolation and RT2 Profiler PCR Array Human Cells Death Pathway Finder:

Human myelogenous leukemia cell lines KG-1, KG-1a, K-562 and THP-1 were used in log phase of growth. Cells were seeded into 25 cm² flask at a density of 2.5 10⁵ cells/ml in the corresponding media supplemented with 2 mM L-Glutamine, 25 mM HEPES, 1% FBS, and 50 μg/ml Gentamicin, with or without treatment with 5 μM MIA-602 for 24 hours. For each cell line, total RNA was isolated from 3.5×10⁶ cells using RNaesy Mini Kit on (Qiagen, Ltd.) by following manufacturer's instructions. The yield and quality of isolated RNA was determined spectrophotometrically using 260 nm and 260/280 nm ratio, respectively. An amount of 0.5 μg of RNA in a final volume of 200 was reverse transcribed into cDNA using RT2 First Strand Kit (Qiagen Ltd.) Human Cell Death Pathway Finder RT2 Profiler PCR Array (SABiosciences, Corp.) was used to analyze gene expression of 84 selected genes involved in different cell death pathways. The qPCR was performed using 30 ng of cDNA per well in a BioRad Real Time PCR System (BioRad) as follows: one cycle of 10 min at 95° C., followed by 40 cycles of 15 sec at 95° C., and 1 min at 60° C. Raw data was analyzed using the web-based automated software for RT2 Profiler PCR Array Data Analysis. Fold-changes in gene expression were calculated using ΔΔCt method. Normalization was performed using five housekeeping genes on the array. Genes that are significantly up- or downregulated by MIA-602 treatment, with at least a 2-fold difference compared to control, are shown. Significance was determined by two-tailed Student's t-test.

Apoptosis Assay:

Apoptosis rates were determined with the terminal deoxynucleotidyl transferase-mediated dUTP nick end labeling (TUNEL) method (DeadEnd™ Fluorometric TUNEL System Protocol, Promega, Madison, Wis., USA) using a fluorescent microscope according to manufacturer instructions. Subsequently the numbers of apoptotic and live cells were determined.

In Vivo Experiments in Mice:

Eight-weeks-old female athymic nude mice (Hsd: Athymic Nude-Foxn1\hat{ }nu), obtained from Harlan Laboratories, were housed in sterile cages in a temperature-controlled room with a 12-hours light\12-hours dark schedule. Mice were fed autoclaved chow and water ad libitum. Human myelogenous leukemia cells KG-1, KG-1a, K-562 and THP-1 were used in log phase of growth. Xenografts were initiated by subcutaneous (s.c.) injection of 1×107 cells into the right flank of nude mice. For each cell line, once tumor reached an appropriate size (approx. 40 mm3), mice were randomly divided into two groups of 15 animals each, which received s.c. injections twice a day into the left flank of, phosphate-buffered saline (PBS) (100 (0.1.1) containing 0.1% DMSO, or MIA-602 at a dose of 10 μg. Tumor volume (length×width×height×0.5236) and body weight were measured every 7 days for a total of 28 days. At the end of the experiment, mice were sacrificed under carbon dioxide followed by cervical dislocation. All animal procedures were performed in compliance with the Guide for the Care and Use of Laboratory Animals published by the NIH, and approved by the Animal Care and Use Committee of the University of Miami.

Western Blot Analysis:

Myelogenous cell lines, primary cells and tumor xenograft tissues were lysed/homogenized in RIPA buffer. Protein concentration was determined using a BCA Protein Assay kit (Thermo Scientific, IL). Equal amounts of proteins were denatured in 4× Laemmli's sample buffer followed by 5 min of boiling, and then resolved on a 4-20% Tris-Glycine gel (Bio-Rad, CA). Proteins were transferred onto Immun-Blot® PVDF (Bio-Rad, CA) or nitrocellulose membrane. Membranes were then blocked in either 5% non-fat dry milk or 5% bovine serum albumin in TBS solution, or 50-50% TBS to Odyssey Blocking Buffer (LICOR Biosciences, Lincoln, Nebr.) for 1 h at room temperature, as appropriate for the subsequent primary and secondary antibody stainings. Blots were incubated overnight at 4° C. with primary antibody. Primary antibodies used were as follows: β-actin (4937; Cell Signaling), GHRH (ab-187512; Abcam), GHRH-R/SV1 (ab-28692; Abcam), p44/42 MAPK (Erk1/2) (9102; Cell Signaling), phospho p44/42 MAPK (Erk1/2) (4370; Cell Signaling), pan AKT (4685; Cell Signaling). Membranes were incubated with Infrared IRDye®-labeled secondary antibodies (1:10000; LI-COR, Lincoln, Nebr.), for visualization with the Odyssey Infrared Imaging System (LI-COR Biosciences, Lincoln, Nebr.). Otherwise, membranes were incubated with horse-radish peroxidase-linked anti-mouse or anti-rabbit antibody (Cell Signaling/Thermo Fisher Scientific) for 1 h at room temperature, and then washed four times in TBS-T. Detection was performed with chemiluminescent reagents (Immune-Star HRP, Bio-Rad, CA) or SuperSignal West Pico Chemiluminescent Substrate, (Thermo Fisher Scientific).

Immunocytochemistry and Visualization of MIA-602 Cellular Uptake:

Cells were counted and harvested by centrifugation, and the pellet was resuspended in fresh medium at 10⁶ cells/mL density. A drop of cell suspension was placed on poly-L-lysine-coated slides for 1 hour, then fixed in 4% paraformaldehyde for 15 minutes. Permeabilization was performed by a 10-minuted incubation in PBS containing 0.2% Triton-X, and cells were then blocked with 5% goat serum in PBS for 1 hour. Cells were incubated for 1 hour with antibodies to GHRH-R (1:150 dilution, Abcam) diluted in PBS with 2% goat serum, followed by 1-hour incubation with a fluorescently-labelled anti-rabbit secondary antibody (Alexa Fluor 488, Jackson Immunoresearch, West Grove, Pa.). Cells were then mounted in Vectashield mounting medium containing DAPI (Vector Laboratories). Images were acquired on a Nikon Eclipse Ti fluorescence microscope with a 20× objective.

For the detection of cellular uptake, MIA-602 was conjugated to Alexa Fluor 555 according to the manufacturer's instructions (Alexa Fluor 555 NHS ester, tris(triethylammonium salt, Life Technologies, Carlsbad, Calif.). Cells were incubated with 2.5 μM fluorescently labelled peptide or unconjugated fluorophore for 30 minutes (1×10⁶ cells/tube). At the end of incubation, cells were washed 3 times in PBS, fixed in 4% paraformaldehyde, mounted, and imaged as described above. ImageJ software (NIH, Bethesda, Md.) was used to recolor the images, for the purpose of enhancing visual perception of cellular uptake.

Immunohistochemistry:

Paraffin sections were deparaffinized and rehydrated with xylene and graded series of ethanol. Antigen retrieval was performed in a pressure cooker with Trilogy antigen retrieval solution (Cell Marque, Rocklin, Calif.) and tissues were blocked with Background Terminator (Biocare Medical, Concord, Calif.). Tissues were incubated with GHRHR antibodies (1:200 dilution, ab76263, Abcam) or GHRH (1:50 dilution, ab187512, Abcam) antibodies overnight.

Anti-rabbit secondary antibody (Alexa Fluor 488; Life Technologies) was applied for 1 hour and slides were counterstained with nuclear staining and were mounted in Slowfade Diamond Antifade Mountant. Images were acquired on a Nikon Eclipse Ti fluorescence microscope.

Patient Samples:

Primary AML samples were collected by the Tissue Banking Core Facility (TBCF) at the University of Miami/Sylvester Comprehensive Cancer Center per IRB-approved protocol (20060858). Bone marrow aspirates were subjected to Ficoll (Ficoll Paque Plus, GE Healthcare Bio-Sciences, Pittsburgh, Pa., USA) density gradient centrifugation to purify the mononuclear fraction.

Statistical Analysis:

For statistical evaluation, GraphPad Prism 6 software (GraphPad Software) was used. Results are expressed as means±SEM. One-way ANOVA followed by Dunnett post-hoc test was used and significance accepted at p<0.05.

The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A method of treating myeloid leukemia, the method comprising administering a GHRH antagonist to mammalian subject in need thereof.

2. The method of claim 1, wherein GHRH antagonist comprises the amino acid sequence (Formula I/SEQ ID NO: 2): R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,
wherein R¹ is PhAc (phenylacetyl), Nac (naphthylacetyl), Oct (octanoyl), N-Me-Aib (N-methyl-alpha-aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl), PhAc-Ada (phenylacetyl 12-aminododecanoyl), Ac-Ada-D-Phe, Ac-Ada-Phe, Dca-Ada(dichloroacetyl-12-aminododecanoyl), Nac (naphthylacetyl), Nac-Ada, Ada-Ada, or CH₃(CH₂)₁₀-CO-Ada;
A⁴ is Ala or Me-Ala;
A⁶ is Cpa (para-chlorophenylalanine) or Phe(F)₅;
A⁸ is Ala, Pal (pyridylalanine), Dip ((3,3-diphenyl)alanine), or Me-Ala;
A¹⁰ is FPa5, Tyr(Alk) where Alk is Me or Et;
A¹¹ is His or Arg; A¹² is Lys, Lys(0-11) (Lys(A0-A1-A2-A3-A4-A5-A6-A7-A8-A9 A10-A11-), Lys(Me)₂, or Orn (ornithine);
A¹⁵ is Abu (alpha-aminobutyric acid) or Orn;
A¹⁷ is Leu or Glu;
A²⁰ is Har (homoarginine) or His;
A²¹ is Lys, Lys(Me)₂ or Orn;
A²⁹ is Har, Arg or Agm (agmatine);
R₂ is β-Ala, Amc (8-aminocaprylyl), Apa (5-aminopentanoyl), Ada (12-aminododecanoyl), AE₂A (8-amino-3,6-dioxaoctanoyl), AE₄P (15-amino-4,7,10,13-tetraoxapentadecanoyl), ε-Lys(α-NH₂) (a Lys residue, the 8-amino group of which is acylated by the carbonyl group of an N-terminally located amino acid; the α-amino group of the Lys residue is free), Agm (agmatine), or absent; and
R³ is Lys(Oct), Ahx (6-aminohexanoyl), or absent.

3. The method of claim 1, wherein the GHRH antagonist is MIA-602, MIA-604, MIA-606, MIA-610, MIA-640, or MIA-690.

4. The method of claim 1, wherein the GHRH antagonist is MIA-602.

5. The method of claim 1, wherein the GHRH antagonist is administered via intradermal, intramuscular, intraperitoneal, intravenous, intraarterial, subcutaneous, intranasal, epidural, sublingual, intranasal, intracerebral, intraventricular, intrathecal, intravaginal, transdermal, rectally, inhalation, or topical delivery.

6. The method of claim 5, wherein the GHRH antagonist is administered subcutaneously.

7. The method of claim 3, wherein the GHRH antagonist is administered via intradermal, intramuscular, intraperitoneal, intravenous, intraarterial, subcutaneous, intranasal, epidural, sublingual, intranasal, intracerebral, intraventricular, intrathecal, intravaginal, transdermal, rectally, inhalation, or topical delivery.

8. The method of claim 7, wherein the GHRH antagonist is administered subcutaneously.

9. The method of claim 4, wherein the GHRH antagonist is administered via intradermal, intramuscular, intraperitoneal, intravenous, intraarterial, subcutaneous, intranasal, epidural, sublingual, intranasal, intracerebral, intraventricular, intrathecal, intravaginal, transdermal, rectally, inhalation, or topical delivery.

10. The method of claim 9, wherein the GHRH antagonist is administered subcutaneously.

11. The method of claim 1, wherein the myeloid leukemia is acute myeloid leukemia.

12. The method of claim 3, wherein the myeloid leukemia is acute myeloid leukemia.

13. The method of claim 4, wherein the myeloid leukemia is acute myeloid leukemia.

14. The method of claim 10, wherein the myeloid leukemia is acute myeloid leukemia.

15. The method of claim 1, wherein the myeloid leukemia is chronic myeloid leukemia.

16. The method of claim 3, wherein the myeloid leukemia is chronic myeloid leukemia.

17. The method of claim 4, wherein the myeloid leukemia is chronic myeloid leukemia.

18. The method of claim 10, wherein the myeloid leukemia is chronic myeloid leukemia.


SEQUENCE LISTING (8 sequences)

SEQ ID NO: 1 - protein, 29 aa, Homo sapiens
  MISC_FEATURE: hGHRH (1-29)HN2
YADAIFTNSYRKVLGQLSARKLLQDIMSR

SEQ ID NO: 2 - protein, 32 aa, Artificial Sequence
  Synthetic Polypeptide
  MISC_FEATURE (1)..(1): Xaa is PhAc (phenylacetyl), Nac (naphthylacetyl), Oct (octanoyl), N-Me-Aib (N-methyl-alpha- aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl),
  MISC_FEATURE (1)..(1): Xaa is PhAc (phenylacetyl), Nac (naphthylacetyl), Oct (octanoyl), N-Me-Aib (N-methyl-alpha- aminoisobutyroyl), Dca (dichloroacetyl), Ac-Ada (acetyl-12-aminododecanoyl), Fer (ferulyl), Ac-Amc (acetyl-8-aminocaprylyl), Me-NH-Sub (methyl-NH-suberyl),
  MISC_FEATURE (3)..(3): Xaa is D-Arg
  MISC_FEATURE (5)..(5): Xaa is Ala or Me-Ala
  MISC_FEATURE (7)..(7): Cpa (para-chlorophenylalanine) or Phe(F)
  MISC_FEATURE (9)..(9): Xaa is Ala, Pal (pyridylalanine), Dip ((3,3- diphenyl)alanine), or Me-Ala
  MISC_FEATURE (10)..(10): Xaa is Har
  MISC_FEATURE (11)..(11): Xaa is FPa5, Tyr(Alk) where Alk is Me or Et
  MISC_FEATURE (12)..(12): Xaa is His or Arg
  MISC_FEATURE (13)..(13): Xaa is Lys, Lys(0-11) (Lys(A0-A1-A2-A3-A4-A5-A6-A7-A8-A9 A10-A11-), Lys(Me)2, or Orn (ornithine)
  MISC_FEATURE (16)..(16): Xaa is Abu (alpha-aminobutyric acid) or Orn
  MISC_FEATURE (18)..(18): Xaa is Leu or Glu
  MISC_FEATURE (21)..(21): Xaa is Har (homoarginine) or His
  MISC_FEATURE (22)..(22): Xaa is Lys, Lys(Me)2 or Orn
  MISC_FEATURE (28)..(28): Xaa is Nle
  MISC_FEATURE (29)..(29): Xaa is D-Arg
  MISC_FEATURE (30)..(30): Xaa is Har, Arg or Agm (agmatine);
  MISC_FEATURE (31)..(31): Xaa is  Ala, Amc, Apa, Ada, AE2A , AE4P,E -Lys( alpha-NH2), Agm, or absent
  MISC_FEATURE (32)..(32): Xaa is  Lys(Oct), Ahx (6-aminohexanoyl), or absent
  MISC_FEATURE (32)..(32): C-Term NH2
XYXDXIXTXXXXXVLXQXSAXXLLQDIXXXXX

SEQ ID NO: 3 - protein, 32 aa, Artificial Sequence
  Synthetic Polypeptide
  MISC_FEATURE (1)..(1): Xaa is Phac
  MISC_FEATURE (2)..(2): Xaa is Ada
  MISC_FEATURE (4)..(4): Xaa is D-Arg
  MISC_FEATURE (8)..(8): Xaa is Phe(F)5
  MISC_FEATURE (11)..(11): Xaa is Har
  MISC_FEATURE (12)..(12): Xaa is Tyr(Me)
  MISC_FEATURE (14)..(14): Xaa is Orn
  MISC_FEATURE (17)..(17): Xaa is Abu
  MISC_FEATURE (23)..(23): Xaa is Orn
  MISC_FEATURE (29)..(29): Xaa is Nle
  MISC_FEATURE (30)..(30): Xaa is D-Arg
  MISC_FEATURE (31)..(31): Xaa is Har
  MISC_FEATURE (32)..(32): Xaa is Agm
  MISC_FEATURE (32)..(32): C-Term NH2
XXYXDAIXTAXXHXVLXQLSAHXLLQDIXXXX

SEQ ID NO: 4 - protein, 32 aa, Artificial Sequence
  Synthetic Polypeptide
  MISC_FEATURE (1)..(1): Xaa is Phac
  MISC_FEATURE (2)..(2): Xaa is Ada
  MISC_FEATURE (4)..(4): Xaa is D-Arg
  MISC_FEATURE (8)..(8): Xaa is Phe(F)5
  MISC_FEATURE (9)..(9): Xaa is Thr(Me)
  MISC_FEATURE (11)..(11): Xaa is Har
  MISC_FEATURE (12)..(12): Xaa is Tyr(Me)
  MISC_FEATURE (14)..(14): Xaa is Orn
  MISC_FEATURE (17)..(17): Xaa is Abu
  MISC_FEATURE (23)..(23): Xaa is Orn
  MISC_FEATURE (29)..(29): Xaa is Nle
  MISC_FEATURE (30)..(30): Xaa is D-Arg
  MISC_FEATURE (31)..(31): Xaa is Har
  MISC_FEATURE (31)..(31): Xaa is Har
  MISC_FEATURE (32)..(32): Xaa is Agm
  MISC_FEATURE (32)..(32): C-Term NH2
XXYXDAIXXAXXHXVLXQLSAHXLLQDIXXXX

SEQ ID NO: 5 - protein, 31 aa, Artificial Sequence
  Synthetic Polypeptide
  MISC_FEATURE (1)..(1): Xaa is Phac
  MISC_FEATURE (3)..(3): Xaa is D-Ala
  MISC_FEATURE (7)..(7): Xaa is Cpa
  MISC_FEATURE (10)..(10): Xaa is Har
  MISC_FEATURE (11)..(11): Xaa is Fpa
  MISC_FEATURE (13)..(13): Xaa is Orn
  MISC_FEATURE (16)..(16): Xaa is Abu
  MISC_FEATURE (22)..(22): Xaa is Orn
  MISC_FEATURE (28)..(28): Xaa is Nle
  MISC_FEATURE (29)..(29): Xaa is D-Arg
  MISC_FEATURE (30)..(30): Xaa is Har
  MISC_FEATURE (31)..(31): Xaa is Ada
  MISC_FEATURE (31)..(31): C-Term NH2
XYXDAIXTAXXHXVLXQLSAHXLLGAIXXXX

SEQ ID NO: 6 - protein, 32 aa, Artificial Sequence
  Synthetic Polypeptide
  MISC_FEATURE (1)..(1): Xaa is Phac
  MISC_FEATURE (2)..(2): Xaa is Ada
  MISC_FEATURE (4)..(4): Xaa is D-Arg
  MISC_FEATURE (8)..(8): Xaa is Cpa
  MISC_FEATURE (11)..(11): Xaa is Har
  MISC_FEATURE (12)..(12): Xaa is Fpa
  MISC_FEATURE (14)..(14): Xaa is Orn
  MISC_FEATURE (17)..(17): Xaa is Abu
  MISC_FEATURE (23)..(23): Xaa is Orn
  MISC_FEATURE (29)..(29): Xaa is Nle
  MISC_FEATURE (30)..(30): Xaa is D-Arg
  MISC_FEATURE (31)..(31): Xaa is Har
  MISC_FEATURE (32)..(32): Xaa is Ada
  MISC_FEATURE (32)..(32): C-Term NH2
XXYXDAIXTAXXHXVLXQQSAHXLLQDIXXXX

SEQ ID NO: 7 - protein, 31 aa, Artificial Sequence
  Synthetic Polypeptide
  MISC_FEATURE (1)..(1): Xaa is Phac
  MISC_FEATURE (2)..(2): Xaa is Ada
  MISC_FEATURE (4)..(4): Xaa is D-Arg
  MISC_FEATURE (8)..(8): Xaa is Cpa
  MISC_FEATURE (11)..(11): Xaa is Har
  MISC_FEATURE (12)..(12): Xaa is Fpa
  MISC_FEATURE (14)..(14): Xaa is Orn
  MISC_FEATURE (17)..(17): Xaa is Abu
  MISC_FEATURE (23)..(23): Xaa is Orn
  MISC_FEATURE (29)..(29): Xaa is Nle
  MISC_FEATURE (30)..(30): Xaa is D-Arg
  MISC_FEATURE (31)..(31): Xaa is Har
  MISC_FEATURE (31)..(31): C-Term NH2
XXYXDAIXTAXXHXVLXQLSAHXLLQDIXXX

SEQ ID NO: 8 - protein, 31 aa, Artificial Sequence
  Synthetic Polypeptide
  MISC_FEATURE (1)..(1): Xaa is PhAc
  MISC_FEATURE (2)..(2): Xaa is Ada
  MISC_FEATURE (4)..(4): Xaa is D-ARg
  MISC_FEATURE (8)..(8): Xaa is Fpa
  MISC_FEATURE (11)..(11): Xaa is Har
  MISC_FEATURE (12)..(12): Xaa is Tyr(Me)
  MISC_FEATURE (14)..(14): Xaa is Orn
  MISC_FEATURE (17)..(17): Xaa is Abu
  MISC_FEATURE (22)..(22): Xaa is Orn
  MISC_FEATURE (29)..(29): Xaa is Nle
  MISC_FEATURE (30)..(30): Xaa is D-Arg
  MISC_FEATURE (31)..(31): Xaa is Har
XXYXDAIXTAXXHXVLXQLSAXKLLQDIXXX